(19) United States
(12) Patent Application Publication
(10) Pub. No.: US 2020/0146966 A1
(43) Pub. Date: May 14, 2020

(54) COSMETIC FORMULATIONS WITH ENHANCED DYE FIXATION AND METHODS AND SYSTEMS FOR PREPARATIONS AND USES THEREOF

(71) Applicant: Gracemink, Inc., Bayside, NY (US)

(72) Inventor: Minkyung Grace Choi, Bayside, NY (US)

(73) Assignee: Gracemink, Inc., Bayside, NY (US)

(21) Appl. No.: 16/684,059

(22) Filed: Nov. 14, 2019

Related U.S. Application Data
(60) Provisional application No. 62/851,401, filed on May 22, 2019.
(60) Provisional application No. 62/760,996, filed on Nov. 14, 2018.

(51) Int. Cl. A61K 8/81; A61K 8/02; B41M 5/50; A61K 8/27; A61K 8/25; A61K 8/42; A61Q 3/02; B41M 3/00; B41J 3/407; A61K 8/34

(52) U.S. Cl. CPC A61K 8/817; A61K 8/0204; B41M 5/502; A61K 8/27; A61K 8/25; A61K 8/42; A61K 2800/43; B41M 3/006; B41J 3/407; A61K 8/345; A61K 2800/87; B41M 2205/40; A61K 2800/805; A61Q 3/02

(57) ABSTRACT

Improved undyed substrate formulations described herein have enhanced dye fixation and colorfastness due to opposite electrostatic charges between the base material and the dye. The substrate or base material is formulated to possess an electrostatic charge through the addition of one or more polar or otherwise charged ingredients. A printer device may be used to apply dye to the substrate formulations in a selected image, pattern, or color to produce a customized cosmetic having enhanced dye retention.

This application claims priority to U.S. Provisional Patent Application Ser. No. 62/760,996, entitled Cosmetic Formulation with Enhanced Dye Fixation, filed Nov. 14, 2018, and U.S. Provisional Patent Application Ser. No. 62/851,401, entitled Cosmetic Formulation with Enhanced Dye Fixation, filed May 22, 2019, the contents of both being hereby incorporated by reference.

### BACKGROUND

The present invention relates to formulations for cosmetics, particularly printable cosmetics, having enhanced dye fixation, as well as method and systems for preparing printable cosmetics using the formulations.

Formulations of printed topical cosmetics are typically comprised primarily of a base material and a colorant. The colorant may be adjusted according to color preference and mixed with the base material to allow for the desired application of color to the skin. In printing cosmetics using adequate fixation or bonding between the dye and the base is important to prevent color bleeding, fading, skin staining, and changes in color after application.

In the current state of the art, in making a colored cosmetic product, very fine solid pigment particles (iron oxides, lake pigments, chromium oxides, manganese violets, ultramarines etc.) are pre-mixed and then added to the base material at approximately 10%-30% by weight. The mixture is then mixed under agitation/osterized/grinded until the final composition is homogenous in color and consistency and no streaking of color is observed. If lake pigments are used, these pigments are prepared through the standard “laking” process prior to the start of manufacturing.

The majority of colorants used in topical cosmetics are typically pigments, which are very fine solid particles (iron oxides, lake pigments, etc), since they do not bleed as much as dyes which are fully dissolvable in water. In cosmetics, lake pigments are typically manufactured from synthetic, azo dyes, coal-tar dyes and used in color cosmetics in order to achieve vibrant, consistent colors. Most natural pigments and iron oxides are not as vibrant and the consistency of color is hard to control. A lake pigment is a pigment made by precipitating a dye with an inert binder, or “mordant,” usually a metallic salt. Lakes are pigments made by absorbing a water soluble dye onto an insoluble, inorganic substrate. There is no chemical bond between the dye and the substrate. The dye simply takes on the insoluble nature of the substrate. Typical substrates are aluminum hydrate and aluminum benzoate. The lake pigments are then mixed into the base/filler material of the cosmetics to make the final product. Color pigments usually make up 15-20% of final product by weight. The lake pigment manufacturing process, however, is not suitable for on-demand custom printing of color cosmetics as it entails a number of steps and long waiting times. Additionally you can only make one pigment at a time with this method. Raw dyes are often not used in color cosmetics as they bleed (i.e., they have poor colorfastness) and stain the skin and are subsequently difficult to remove. However, dyes are desirable for use in printing cosmetics as many inkjet devices are compatible with dye-based ink. Adequate fixation or bonding between the dye and the base is important to prevent color bleeding, fading, skin staining, and changes in color after application

In the manufacture of color cosmetics using “printing,” a dye is used to color the base material uniformly such that little to no mixing steps are needed and risk of color streaking from unblended pigments are eliminated. Printable cosmetic formulations allow for individual selection of desired colorants and dyes which are then applied to or otherwise printed on a substrate containing the base material. This enables a significant reduction in time, labor and cost required in manufacturing a final colored composition, and more specifically reduces the time, labor, and cost required to manufacture a variety of colored compositions, or designs. Thus, the colorants and dyes must be customizable, sprayable or printable, easy to work with, safe, mild, and capable of binding to the base with minimal processing steps. Ensuring a strong bond between the colorant and the base is of particular concern. There is no current solution to eliminate or reduce the fugitive properties such as color bleed or skin/nail staining of the dye from base materials. This is an important issue to solve as in order to be considered a realistic and competitive cosmetic product, ordinary wear should not cause the printed cosmetic to dissociate, bleed, or unravel. Further, all ingredients must be permitted for use under relevant regulatory organizations such as the Food and Drug Administration.

Also in the field of printable cosmetics, U.S. Pat. No. 9,498,974, incorporated herein by reference, is notable. This patent describes a device for producing a cosmetic composition in the form of a printer that is modified to receive and process cosmetic components. Colorants are stored in replaceable cartridges and applied by the printer to a substrate containing a base material. Accordingly, the device acts in a similar fashion to an inkjet printer where colorants are stored in cartridges and the base or substrate material (i.e. paper) is external. Other printable cosmetic devices use a dosing or portioning method in which all base materials are premixed into color cartridges and there are no blank external substrates.

International application WO 2015/186583 describes a printed makeup product including multiple different layers, including a base sheet, an inorganic solid layer on the base sheet, where the inorganic solid layer must have a rough surface formed by adhering and then removing a fiber sheet, an inkjet printed image on the inorganic solid layer, and a makeup material on the inkjet printed image. This publication is not concerned with printable makeup formulations for application to the skin as it is with forming realistic printed images, including those with makeup shown on an image of the face, and the complexity of the ingredients, layers and the steps for preparing such formulations are not suitable for the present applications.

Other methods for creating colorfast pigments have also been explored. One method is to create a lake pigment. A lake pigment is a pigment made by precipitating a dye with an inert binder or mordant, which may be a metallic salt. Lake pigments are frequently used in the cosmetics industry and are effective at creating colorfast pigments. However, they are cumbersome and time-consuming as they require precipitation of the pigment followed by drying. These steps are not compatible with on-demand, easy to use printable cosmetic formulations. Further, even lake pigments continue to demonstrate fugitive properties.

International publication WO 2014/135915 created a colorfast, bleed-resistant solution for makeup formulations, particularly for harsh environments like nail polish. A water-soluble organic dye is mixed with a resin and a water-soluble metallic salt and the resin is cross-linked, followed by grinding to a powder. Cross-linking resins and grinding are not compatible with on-demand, easy to use printable cosmetic formulations. Adding the capability to grind to the printer device would make the machine expensive, cumbersome, and difficult to clean. The time consuming nature of the process would not make it very attractive to users.

What is needed, therefore, is an improved formulation or a means for enhancing the bond or fixation between a dye or colorant and a base substrate material that will permit both the colorant and the substrate to be used effectively in an on demand, customizable, easy to use printable cosmetic formulation. Little to no grinding/mixing/blending of the colorant into the base material should be required.

### SUMMARY

The present invention relates to improved methods and formulations for cosmetics, where dyes bond with higher effectiveness and colorfastness to substrate or base materials. In particular, the present invention relates to the introduction of charged ingredients to the substrate or base material and/or to the dye material such that the substrate and the dye possess at least a minimal opposite charge in order to facilitate and improve dye fixation and retention in the substrate. In traditional manufacturing the colorants would sit separately and need to be mixed/grinded/blended for even color development, otherwise there would be streaking. In the present “dye printing” methods the base absorbs the dye and color evenly and therefore doesn't need such mechanical mixing to develop an even result and avoids streaking.

Most untreated raw cosmetic base ingredients exhibit poor dye fixation and bleeding will be observed when water is dropped onto the colored compositions. Similarly, when certain raw base materials have generally the same charge as the dye material, they may not absorb any ink at all. Introducing a charge to the substrate that is opposite to the polarity of the dye assists in binding the ink ingredients by electrostatic forces. This improves dye fixation and reduces dye bleeding and separation.

In many instances, dyes that are suitable for use in cosmetic formulations, i.e. dyes that are approved for use on human skin under applicable regulations, are already polar in nature. Accordingly, in certain embodiments described herein, only the substrate material is treated to provide a charge that is opposite to the charge on the dye. However, in other embodiments, both the dye and the substrate may be treated to introduce opposite electrostatic charges in order to facilitate improved dye fixation. Importantly, all materials used in the dye and the substrate must be safe for use, approved under relevant regulations, and, in certain preferred embodiments, suitable for use in a printing device for on demand printable cosmetics.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a carrier for substrate or base material, in accordance with preferred embodiments described herein.

FIG. 2 shows a carrier for substrate or base material, in accordance with preferred embodiments described herein

FIG. 3 shows a sheet on which substrate or base material is deposited, in accordance with preferred embodiments described herein.

FIG. 4A shows a sheet on which a layer of hydrophobic material and a substrate or base material are deposited, in accordance with preferred embodiments described herein.

FIG. 4B shows a sheet on which portions of hydrophobic material and a substrate or base material are deposited, in accordance with preferred embodiments described herein.

FIG. 5 shows a carrier on which substrate or base material is deposited, in accordance with preferred embodiments described herein.

FIG. 6 shows a carrier on which substrate or base material is deposited and a protective sleeve or envelope, in accordance with preferred embodiments described herein.

FIG. 7 shows a printer device for use with a printer cartridge and substrate carrier, in accordance with preferred embodiments described herein.

FIG. 8 shows a transfer sheet on which dye material is deposited, in accordance with preferred embodiments described herein.

### DETAILED DESCRIPTION OF PREFERRED EMBODIMENTS

One aspect of the current disclosure pertains to an improved cosmetic formulation in which binding of the dye material to the substrate or base material is enhanced, to improve colorfastness and reduce dye bleeding.

Certain preferred embodiments described herein relate to an improved substrate or base material for use in cosmetics. The substrate or base material is formulated to possess an electrostatic charge through the addition or one or more polar or otherwise charged ingredients. The substrate or base material includes only mild, safe, and approved ingredients for use on skin. Accordingly, preferred embodiments described herein include methods for improving dye fixation in a cosmetic formulation which comprise the step of introducing a cationic charge to the base material through the inclusion of cationic ingredients which are themselves safe and approved for use in cosmetics. As consistency, mix ability, and texture are also important to cosmetic formulations, the embodiments described herein must include components that do not lead to unworkable cosmetic formulations.

In additional preferred embodiments, the substrate material is provided with a positive or cationic charge through the addition of one or more cationic ingredients. The cationic ingredients may be one or more cationic ammonium ingredients. The cationic ammonium ingredients can be quaternary ammonium compounds or polycationic polymers. Quaternary ammonium compounds (including polyquaternium-6, which is a polycationic polymer), are cationic molecules and are positively charged, regardless of pH. Preferred examples of the cationic ingredients include, without limitation, polyquaternium-6, polyquaternium-7, polyquaternium-10, quaternium-31 (dicetyldimonium chloride, isopropyl alcohol), cetrimonium chloride, polyquaternium-51, guar hydroxypropyltrimonium chloride, cocodimonium hydroxypropyl hydrolyzed rice protein, and combinations thereof. These ingredients possess generally positive or cationic charges and are approved for use in cosmetic formulations

In further preferred embodiments, the substrate material is provided with a positive or cationic charge through the addition of one or more modified cationized clay compounds. Preferred examples of the modified cationized clay compounds include disteardimonium hectorite, quaternium-18 hectorite, stearalkonium bentonite, quaternium-90 bentonite, and mixtures thereof. These ingredients possess generally positive or cationic charges and are approved for use in cosmetic formulations.

Modified cationized clay compounds are generally the products of reactions of an ammonium salt with a smectite clay. They are generally synthesized by grafting cationic surfactants to clay, such as hectorite (i.e., exchanging the interlayer sodium cations with a cationic surfactant). These cationic surfactants are quaternary ammonium compounds with the template formula [(CH3)3NR]+, [(CH3)2NRR′]+, and [CH3NRR′R″]+, where R, R′, and R″ are alkyl or arylalkyl hydrocarbons. For instance, in stearalkonium bentonite some of the inorganic cations of bentonite have been replaced by [(CH3)2NRR′]+, where R and R′ are an octadecyl alkyl chain (i.e., stearyl group) and a benzyl group, respectively. The exchange is typically performed by the addition of the appropriate alkonium chloride (e.g., stearalkonium chloride) to an alcohol/water slurry of the clay. In the case of disteardimonium hectorite at least some of the sodium cations of hectorite have been exchanged for the [(CH3)2NRR′]+ cation, wherein R and R′ are both octadecyl alkyl chains (i.e., stearyl groups). This exchange is typically carried out by the addition of disteardimonium chloride to an alcohol/water slurry of hectorite. The major by-products are inorganic chlorides (e.g., sodium chloride), which are removed during processing. This cation exchange shifts the nature of these minerals from hydrophilic to lipophilic.

Additional preferred embodiments utilize a combination of one or more cationic ammonium ingredients and one or more modified cationized clay compounds to impart a positive or cationic charge to the base or substrate material.

In preferred embodiments, polyquaternium-6 is used as at least one of the cationic ammonium ingredients added to the substrate or base material. Polyquaternium-6 is a highly charged cationic homopolymer of diallyldimethyl ammonium chloride. In additional preferred embodiments, a combination of polyquaternium-6 and a modified cationized clay compound is used as a cationic ingredient for addition to the substrate or base material. The modified cationized clay compound can be quaternium-18 hectorite, disteardimonium hectorite, stearalkonium bentonite, or combinations thereof, in additional preferred embodiments.

In additional preferred embodiments, the substrate material is provided with a positive or cationic charge through the addition of one of more ionized salts having an cation from Groups 2, 3 or 4 of the periodic table and a +2, +3, or +4 charge, and an appropriate anion. Preferred examples of the cation of the ionized salts are shown below in Table 1.

TABLE 1

| | |
|---|---|
| Group 2: | |
| Barium | Ba + 2 |
| Calcium | Ca + 2 |
| Chromium(II) | Cr + 2 |
| Copper(II) | Cu + 2 |
| Iron(II) | Fe + 2 |
| Lead(II) | Pb + 2 |
| Magnesium | Mg + 2 |
| Manganese(II) | Mn + 2 |
| Mercury(I) | Hg2 + 2 |
| Mercury(II) | Hg + 2 |
| Strontium | Sr + 2 |
| Tin(II) | Sn + 2 |
| Zinc | Zn + 2 |
| Group 3: | |
| Chromium(III) | Cr + 3 |
| Aluminum | Al + 3 |
| Iron(III) | Fe + 3 |
| Manganese(III) | Mn + 3 |
| Group 4: | |
| Tin(IV) | Sn + 4 |

In preferred embodiments, the ionized salt is magnesium carbonate or barium sulfate, or a calcium salt. These salts performed very well in testing for dye fixation & retention. Magnesium, barium, and calcium, in addition to the other Group 2 cations identified above, have a +2 cation charge when ionized. The chemical formula of barium sulfate is BaSO₄ and its molar mass is 233.43 g/mol. It is a salt composed of the barium cation (Ba2+) and the sulfate anion (SO₄²⁻), in which sulfur is attached to four oxygen atoms. The barium metal is in the +2 oxidation state. Magnesium carbonate is a magnesium salt with formula MgCO₃ and its molar mass is 84.3139 g/mol. Magnesium carbonate crystallizes in the calcite structure where in Mg²⁺ is surrounded by six oxygen atoms. It is composed of the magnesium cation (Mg²⁺) and the carbonate anion (CO₂³⁻). Structurally, magnesium and barium (and all Group 2 elements) have in common an outer s-orbital which is full; that is, this orbital contains its full complement of two electrons, which these elements readily lose to form cations with charge +2, and an oxidation state of +2. Suitable anions can be sulfate anions, carbonate anions, and any anions that form an ionized salt with the preferred anions that is safe for use in cosmetic formulations.

Preferred embodiments of the substrate material also include one or more traditional substrate or base components which perform well with the addition of the charged or cationic ingredients, including but not limited to mica, titanium dioxide (anatase or rutile), magnesium stearate, zinc stearate, magnesium myristate, magnesium hydroxide, myristic acid, zinc oxide, silica, boron nitride, trihydroxystearin, bentonite, and combinations thereof. In further preferred embodiments, the substrate or base material includes rutile titanium dioxide, anatase titanium dioxide, oil dispersible (or oil soluble) titanium dioxide, or titanium dioxide coated with magnesium myristate, where the coating of magnesium myristate makes up about 2.5% to about 3.5% by weight of the titanium dioxide/magnesium myristate particles, with the remaining 97.5% to 96.5% being the titanium dioxide. In additional preferred embodiments, magnesium myristate may be used as a coating for other base materials, such as mica, in similar weight percent amounts. In certain embodiments, a magnesium myristate treatment may be applied by the addition of myristic acid and magnesium hydroxide. In further preferred embodiments, the substrate or base material includes at least one magnesium-containing component or titanium dioxide or mica, or a combination of two of these. In additional preferred embodiments, the base material includes a combination of titanium dioxide, mica, and a magnesium-containing component.

In additional preferred embodiments, a cationic ammonium ingredient is included in the base material by mixing the base material with a solution containing the cationic ammonium ingredients in distilled water at a concentration ranging from about 0.5% to about 4% by weight of the solution, and more preferably at a concentration ranging from about 0.75% to about 3% by weight of the solution.

In additional preferred embodiments, with regard to the use of modified cationized clay compounds such as disteardimonium hectorite, quaternium-18 hectorite, stearalkonium bentonite, quaternium-90 bentonite, and mixtures thereof, these components may make up the bulk of the base material, or a concentration ranging from about 80% to about 100% of the base material by weight. Additional cationic treatment is not required. Modified clay compounds are not typically favored as the sole ingredient for a cosmetic formulation, however, due to color changes that occur in the applied dyes, as well as a lack of vibrancy in the resulting cosmetic. The color is unvibrant because the modified clay material is sheer and not opaque. Accordingly, it is preferred that a base material including primarily a modified clay compound should also include one or more additional ingredients to reduce color changes and increase color vibrancy. This supplemental base material for use with the modified cationized clay compounds can include mica, titanium dioxide (anatase or rutile), magnesium stearate, zinc stearate, magnesium myristate, magnesium hydroxide, magnesium stearate, myristic acid, zinc oxide, silica, boron nitride, trihydroxystearin, whiteners such as arrowroot powder, corn starch, or calcium carbonate, and combinations thereof. The supplemental base material may be added to the modified cationized clay compound in an amount of about 5% to about 30% by weight of the total base material. In certain preferred embodiments, disteardimonium hectorite is included as a modified cationized clay compound in combination with cyclopentasiloxane and a specially denatured alcohol such as SD Alcohol 40-B. These additional components improve the ability of the disteardimonium hectorite to be incorporated into and function as part of a cosmetic formulation.

In additional preferred embodiments, one or more modified cationized clay compounds such as disteardimonium hectorite, quaternium-18 hectorite, stearalkonium bentonite, quaternium-90 bentonite, and mixtures thereof may be added to the base material in an amount of about 40% to about 80% by weight, and the base material is then mixed with a solution containing one or more additional cationic ammonium ingredients in a concentration of about 0.5% to about 4% by weight of the solution, and more preferably at a concentration ranging from about 0.75% to about 3% by weight of the solution.

In preferred embodiments, a solution containing one or more cationic ammonium ingredients in a compatible evaporating/volatile solvent, such as distilled water, is added to the base material in an amount that is sufficient to from a slurry, or to make the base material have a liquid consistency in order to facilitate deposition on a substrate. In preferred embodiments, the solution is added to the base material in an amount of about 30% to about 70% of the total mixture by weight. The base material is allowed to dry prior to use.

Additional preferred embodiments may incorporate one or more emulsifiers into the base material. Emulsifiers are typically soluble in water but not oils. Certain preferred examples of emulsifiers include polysorbate 20, an emulsifying agent including laurate esters of sorbitol where the monoester is condensed with ethylene oxide (polyoxyethylene-20 sorbitan monostearate), and polysorbate 80, an emulsifying agent including sorbitol, ethylene oxide, and oleic acid (polyoxyethylene-20 sorbitan monooleate). Emulsifiers are optional and are not shown to enhance dye fixation in the absence of cationic ingredients.

Preferred embodiments of the cosmetic formulation described herein can use any suitable dye material. Suitable dyes include natural dyes, synthetic colorants, coal tar, FDA-approved cosmetic color additives (e.g., 21 C.F.R. Part 73, Subpart C, 21 C.F.R. Part 74 Subpart C—Cosmetics, 21 C.F.R. Part 82 Subparts B, C, and D), or any other substances that can cause a change in the color of the base material and are suitable for use in cosmetics. Certain preferred embodiments utilize dye material having an anionic or negative charge that are safe for use in cosmetics, including approved edible inks. These dyes may include one or more of water, propylene glycol, glycerin, carmoisine, polysorbate 80, sodium hydroxide, mono and di-glycerides, potassium citrate, methyl paraben, FD&C dyes such as Red No. 3, Red No. 40, Blue No. 1, Yellow No. 5, and Yellow No. 6, and combinations thereof. In additional preferred embodiments these dyes are suitable for use in conjunction with printable cosmetics and can be dispensed from a suitable printing device. In additional preferred embodiments the dye material is formulated to have a desired electrostatic charge, either anionic or cationic, through the inclusion of appropriate ingredients.

The present cosmetic formulations may be in the form of powders, solids, creams, liquids, and the like. In preferred embodiments, the base material is kept separate from the dye material until the user selects the desired shade of the cosmetic. The appropriate dye material (to produce the selected shade) is then added to, mixed with, or otherwise printed or stamped on the base material. This may be accomplished manually, through the use of a brush, marker, or pen, or through the use of an appropriate printing or stamping device, including those that can be implemented in a home or in a retail store or kiosk environment.

In preferred embodiments described herein, the cosmetic formulations are suitable for use as eye shadows, blush, face powder, or other similar cosmetics. Further preferred embodiments include the use of base materials suitable to form customizable nail polishes, lipsticks, lip glosses, foundation, mascara, eyeliner and other similar cosmetics. Suitable base materials for the production of customizable nail polishes, lipsticks, lip glosses include clear nail polish, uncolored lip gel, and the like. Compatible uncolored base materials may also be added/mixed after printing the color/dye fixation to base step, in order to create the final product (nail polish, lip gloss etc.). Generally, the cosmetic formulations described herein are capable of use in any cosmetic product regardless of consistency, formulation, or area of intended use. While the base materials for these cosmetics may vary from what is described herein, the concepts are the same. A cationic or positive charge may be introduced to the base material in order to enhance dye fixation.

Preferred embodiments described herein include base formulations for preparing colored nail polish. Using appropriate nail base materials, colored nail polish can be prepared by using the same concepts described herein. In other words, a cationic or positive charge may be introduced into the base material in order to enhance dye fixation. Conventional nail polish typically consists of a polymer, most commonly nitrocellulose, dissolved in a solvent, which is usually ethyl acetate or butyl acetate. When it is applied the solvent evaporates, leaving the polymer to form a film on the nail. Adhesive polymer resins that are also contained within the formulation help the polymer film to stick to the nail. These so-called film modifiers also impart a glossiness to the polymer finish. Gel nail polish is an alternative formulation which consists of methacrylate compounds and photoinitiating compounds such as benzoyl peroxide. Unlike conventional nail polish, these mixtures aren't simply applied and left to dry. Instead they are applied in layers which are exposed to ultraviolet light. This kicks off a polymerisation process which solidifies the polish. Shellac is a hybrid of gel and nail polish that is cured and hardened with a UV light. It is less hard than gel and soaks off with acetone.

Nail polish formulations described herein according to preferred embodiments can use any type of nail polish base. These include conventional (solvent-based), gel, shellac (photoinitiated), acrylic, dip powder, water(aqua)-based, solvent-free, and stains. Preferred formulations are solvent-free, water(aqua)-based nail formulations and the like. Conventional nail polish ingredients often make it difficult for uniform mixing of inks without agitation and in some instances change the color of certain dyes. It is important to select base materials that are easily miscible with the cosmetic ink (particularly its solvents propylene glycol, water, etc.) to ensure uniform coloring with little to no mixing needed.

Preferred base ingredients for a nail polish formulation according to preferred embodiments herein include cosmetic grade film formers, polymers, resins, and the like. Film formers include PVP, acrylates, acrylamides, methacrylates, polyurethane and various copolymers. Preferred film forming ingredients include but are not limited to, cationic acrylic polymer, styrene/acrylates/ammonium methacrylate copolymer (gloss film), ammonium styrene/acrylates copolymer, polyquaternium-91 and polyacrylate-15, styrene acrylates copolymer, acrylates copolymer, polyethylene, polyvinyl alcohol, polyvinyl acetate, acrylic copolymer, acrylate copolymer/styrene-acrylate copolymer, polyurethane-2, and trimethylsiloxysilicate. Non-film forming ingredients can include water, denatured alcohol, propylene glycol n-butyl ether, dipropylene glycol dibenzoate, neem oil, polyethylene, rheology modifiers, slip additives, silicones, wetting agents, fillers, antifoams, chelating agents, dispersants, preservatives, thickeners, UV screens, surfactants, therapeutic and prophylactic agents (actives), moisturizers, perfumes, neutralizing agents, antioxidants, additional film-forming polymers and non-film-forming polymers.

As in other preferred embodiments described herein, a cationic or positive charge may be introduced to the nail polish base material in order to enhance dye fixation and prevent staining of dye on the nail and skin. Any suitable cationic ingredient can be used. Some preferred cationic additives include polyquaternium-6, stearalkonium bentonite, disteardimonium hectorite, and stearalkonium hectorite.

One preferred embodiment of a nail polish base formulation includes water, polyacrylate-42, dibutyl sebacate, PPG-2 methyl ether, oxidized polyethylene, phenylpropanol, caprylyl glycol, decylene glycol, and one or more cationic ingredients as described herein. An additional preferred embodiment of a nail polish base formulation includes water, polyacrylate-42, dipropylene glycol dibenzoate, PPG-2 methyl ether, oxidized polyethylene, phenylpropanol, caprylyl glycol, decylene glycol, and one or more cationic ingredients as described herein. An additional preferred embodiment of a nail polish base formulation includes water, polyacrylate-42, acetyl tributyl citrate, dibutyl sebacate, phenylpropanol, caprylyl glycol, decylene glycol, and one or more cationic ingredients as described herein. An additional preferred embodiment of a nail polish base formulation includes water, polyacrylate-42, dibutyl sebacate, PPG-2 methyl ether, oxidized polyethylene, phenylpropanol, caprylyl glycol, decylene glycol, and one or more cationic ingredients as described herein.

Additional preferred embodiments of a nail polish base formulation may include ingredients that make up either a clear base or a white opaque base. Preferred embodiments of a clear base can include about 93 weight percent of a mixture of water, polyacrylate-42, dibutyl sebacate, PPG-2 methyl ether, oxidized polyethylene, phenylpropanol, caprylyl glycol, and decylene glycol, about 5 weight percent of additional water, and about 2 weight percent of the one or more cationic ingredients described herein. Preferred embodiments of a white opaque base can include about 94.5 weight percent of a mixture of water, polyacrylate-42, dibutyl sebacate, PPG-2 methyl ether, oxidized polyethylene, phenylpropanol, caprylyl glycol, and decylene glycol, about 3.5 weight percent of a mixture of water, ammonium acrylates copolymer, CI 77891, methylpropanediol, simethicone, caprylyl glycol, and phenylpropanol, and about 2 weight percent of the one or more cationic ingredients described herein.

Preferred embodiments described herein also include base formulations for creating colored mascara, eyeliner, eyeshadow, and eyebrow products. Preferred base formulations for these products utilize similar ingredients as nail polish formulations. In other words, the preferred formulations for mascara, eyeliner, eyeshadow, and eyebrow products include cosmetic grade film formers, polymers, resins, and the like, plus one or more cationic ingredients as described herein to impart a positive charge to the base material in order to enhance dye fixation.

Preferred embodiments described herein also include base formulations for preparing colored lip and cream based cosmetics, such as cream eye shadow, cream blush, and the like. Conventional lipsticks commonly include wax to give structure/rheology to the product as well as water-resistance. However, waxes make it difficult for uniform mixing of inks without agitation. It is important to formulate a base material that is easily miscible with the cosmetic ink (as well as its solvents propylene glycol, water, and the like) to ensure uniform coloring and little to no mixing needed. If the solvents used with the cosmetic ink change then the base materials should be adjusted accordingly.

One alternative to wax/non-miscible ingredients for lip base materials include stearalkonium bentonite, disteardimonium hectorite, and stearalkonium hectorite gels because they not only act as a rheology modifier but also as a cationic ingredient needed for color retention. They exhibit water-proofing characteristics. Further water-proofing can be enhanced by the addition of cosmetic film-formers and polymers such as those described above. In general, and particularly for ink solvents that contain water and propylene glycol, lip bases that include water, such as lip powders, work well also. Some of these water including formulas also include silica silate and silica.

A preferred embodiment of a formulation for lip and cream based makeup that includes a red dye includes dimethicone, dimethicone/vinyl dimethicone crosspolymer, water, cyclopentasiloxane, polyglyceryl-2 triisostearate, glycerin, red 7 (CI 15850:1), pentylene glycol, cetyl peg/ppg-10/1 dimethicone, butyl acrylate/hydroxypropyl dimethicone acrylate copolymer, methyl trimethicone, phenoxyethanol, silica, caprylyl glycol, glyceryl acrylate/acrylic acid copolymer, 1,2-hexanediol, ethylhexylglycerin, propanediol, PEG/PPG-18/18 dimethicone, silica dimethyl silylate, and methicone. One or more cationic ingredients described herein may be included in the formulation to prevent staining, if staining should be avoided. Some cosmetic formulations, such as lip stain, are intended to stain the skin and may not require the addition of cationic ingredients.

An additional preferred embodiment of a base formulation for colored lip and cream based makeup includes water, ethylhexyl palmitate, butylene glycol, glycerin, pentylene glycol, hydroxyethyl acrylate/sodium acryloyldimethyl taurate copolymer, squalane, phenoxyethanol, ethylhexyl methoxycinnamate, hydrogenated polydecene, polysorbate 60, ethylhexylglycerin, sorbitan isostearate, disodium EDTA, tetrasodium EDTA, polymethylsilsesquioxane, methylparaben, and silica. An additional preferred embodiment of a formulation for lip and cream based makeup includes water, dimethicone, octyldodecanol, isododecane, butylene glycol, alcohol, acrylates/polytrimethylsiloxymethacrylate copolymer, cetyl PEG/PPG-10/1 dimethicone, trimethyl pentaphenyl trisiloxane, disteardimonium hectorite, polyglyceryl-4 isostearate, magnesium sulfate, phenoxyethanol, propylene carbonate, synthetic fluorphlogopite, PEG/PPG-18/18 dimethicone, alumina, disodium stearoyl glutamate, aluminum hydroxide, linalool, and pentaerythrityl tetra-di-t-butyl hydroxyhydrocinnamate. An additional preferred embodiment of a formulation for lip and cream based makeup includes water, polyurethane-35, xylitol, polyglyceryl-2 caprate, glycerin, butylene glycol, tri-C12-13 alkyl citrate, methylpropanediol, polysorbate 80, PEG-60 hydrogenated castor oil, panthenol, phenoxyethanol, fragrance, caprylyl glycol, tocopherol, palmitic acid, ethylhexylglycerin, trisodium edta, tromethamine, phenylpropanol, and myristic acid. To the extent these preferred formulations do not already include cationic ingredients, one or more cationic ingredients can be added to prevent staining, if staining should be avoided.

A preferred embodiment of a base formulation for colored lip powder includes water, glycerin, propanediol, silica silylate, sodium benzoate, potassium sorbate, niacinamide, natto gum, saussurea involucrata extract, bambusa vulgaris leaf/stem extract, vaccinium angustifolium (blueberry) fruit extract, tocopheryl acetate, Cocos nucifera (coconut) oil, and hydrolyzed hyaluronic acid. To the extent this preferred formulation does not already include cationic ingredients, one or more cationic ingredients can be added to prevent staining, if staining should be avoided.

In further preferred embodiments, the base material may be encapsulated in whole or in part within the polar or cationic ingredients.

For all preferred embodiments of base formulations, it is important to select base materials that are easily miscible with the cosmetic ink. Preferred examples of the cosmetic ink referred to herein include solvents such as propylene glycol, water, and the like. This ensures uniform coloring with little to no mixing needed when the ink is deposited onto the base in the carrier. If the solvents in the ink change then the base materials should be adjusted accordingly to use similar materials (i.e. “like into like”). The addition of cationic ingredients as described herein is useful for all cosmetic formulations and can be included in any base material for such formulation. Some require a lot more agitation or mixing than others due to the miscibility of the ink and the base ingredients. For example nitrocellulose in nail polish and wax in lipstick may make the base difficult to mix. In these instances it is best to find an alternative. formulation that readily accepts the ink so that the base is readily colored as the ink is dropped onto the base in the carrier.

Preferred embodiments of the colored cosmetic formulations described herein may include dyes in the ink. Some dyes are susceptible to light/UV exposure which can compromise the color stability of the finished cosmetic composition (i.e. color changing/fading). Several preferred options are available to delay or prevent color instability. First, cosmetic stabilizers may be added. These may include one or more of pentaerythrityl tetra-di-t-butyl hydroxyhydrocinnamate, sodium benzotriazolyl butylphenol sulfonate, buteth-3 and tributyl citrate, diethylhexyl syringylidenemalonate, caprylic/capric triglyceride, tris(tetramethylhydroxypiperidinol) citrate with water and alcohol, tetrasodium EDTA (ethylenediaminetetraacetic acid tetrasodium salt), sodium gluconate, and butylated hydroxytoluene.

In a second option, sunscreen agents may be added to the base formulation to prevent color instability of the dyes. These sunscreen agents may include one or more of butyl methoxydibenzoyl methane, benzophenone-3, benzophenone-4, 3,3,5-trimethylcyclohexyl salicylate, octocrylene, and octyl methoxycinnamate.

In a further option, treated or specialty particles may be used in the base formulation to delay color fading. These specialty particles may include amino-acid silane/cationic silane/amino-silane treated particles such as mica with triethoxysilylpropyl acetyl hydroxyprolinate. Other specialty particles could include urethane based powders, such as HDI/trimethylol hexyllactone crosspolymer with methyl methacrylate crosspolymer, or HDI/trimethylol hexyllactone crosspolymer with silica. In one preferred embodiment, a sample formulation containing treated particles (an amino-acid silane treatment) includes both dry ingredients and wet ingredients. The dry ingredients could include about 90 weight percent mica with triethoxysilylpropyl acetyl hydroxyprolinate, about 10 weight percent titanium dioxide with triethoxysilylpropyl acetyl hydroxyprolinate, and an optional color stabilizer in an amount of less than or about 1 weight percent of the dry ingredients. The wet ingredients could include about 96.5 weight percent water, about 2 weight percent P6, about 1 weight percent glycerin, and about 0.5 weight percent of a combination of phenethyl alcohol with glycerin and caprylhydroxamic acid, a preservative. In a preferred embodiment, 0.6 g of the exemplary dry ingredients are mixed with 2.4 g of the exemplary wet ingredients.

In a further option, color fading of dyes is decreased by limiting the percentage of titanium dioxide. Titanium dioxide is known to increase fading of certain dyes. Thus its presence in the formulation should be limited to less than about 10 weight percent.

In preferred embodiments, a base formulation that resists color fading can include dry ingredients and wet ingredients. The dry ingredients preferably include (by weight of the dry ingredients) about 90-100% filler, which is preferably mica or mica that has been treated to enhance color stability, about 1% or less color stabilizer, which can preferably be sodium gluconate or pentaerythrityl tetra-di-t-butyl hydroxyhydrocinnamate, optionally about 10% or less opacifier, which may be titanium dioxide that is preferably treated, and optionally about 10% or less urethane powder, which may include a crosspolymer of hexamethylene diisocyanate (HDI) and trimethylol hexyllactone and a crosspolymer of methyl methacrylate. The wet ingredients preferably include (by weight) about 96.5% water, 2% P6 (a cationic ingredient), 1% glycerin, and 0.5% of a preservative, which can preferably be a mixture of phenethyl alcohol with glycerin and caprylhydroxamic acid. The base formulation is preferably prepared by mixing 0.6 g of the dry ingredients with 2.4 g of the wet ingredients, at about a 1:4 ratio. The resulting slurry is then preferably distributed thinly on a film or a carrier sheet and allowed to dry. Once the dried base material is printed with ink, it is more likely to retain its color following light exposure.

In additional preferred embodiments, a modified undyed substrate material for use in cosmetics includes a polar ingredient that is a cationic ammonium ingredient. The cationic ammonium ingredient is preferably polyquaternium-6. The substrate material further includes a base material comprising mica and a preservative. Preferably, the mica in the base material is in combination with, or treated with, a material that delays color fading, preferably triethoxysilylpropyl acetyl hydroxyprolinate. The preservative is preferably a combination of phenethyl alcohol, caprylhydroxamic acid, and glycerin. The undyed substrate material can be prepared by mixing dry ingredients with water. All water evaporates after drying. In preferred embodiments, to prepare the substrate material, a mixture is prepared containing about 70-90% by weight, and preferably about 80% by weight, of a combination of mica and triethoxysilylpropyl acetyl hydroxyprolinate, where the combination includes mica in about 98-99% by weight and triethoxysilylpropyl acetyl hydroxyprolinate in about 1-2% by weight. The mixture made to prepare the substrate material preferably further contains about 10% to about 30% by weight, and preferably about 20% by weight, of a solution that includes water and about 1% to about 3%, preferably about 2%, of polyquaternium-6 by weight and about 1% of a preservative by weight. The water can be deionized water, distilled water or regular tap water. The preservative is preferably made up of about 42-60% phenethyl alcohol, by weight, about 12-18% caprylhydroxamic acid, by weight, and about 28-40% glycerin, by weight. After mixing, the slurry will contain about 0.05% to about 0.90% polyquaternium-6 by weight and about 0.10% to about 0.30% preservative by weight, with the remainder being the water mixed with the combination of mica and triethoxysilylpropyl acetyl hydroxyprolinate. After drying, the substrate material will contain about 0.06% to about 1.26% polyquaternium-6, by weight, and about 0.11% to about 0.42% of a preservative, by weight, with the rest being the combination of mica and triethoxysilylpropyl acetyl hydroxyprolinate.

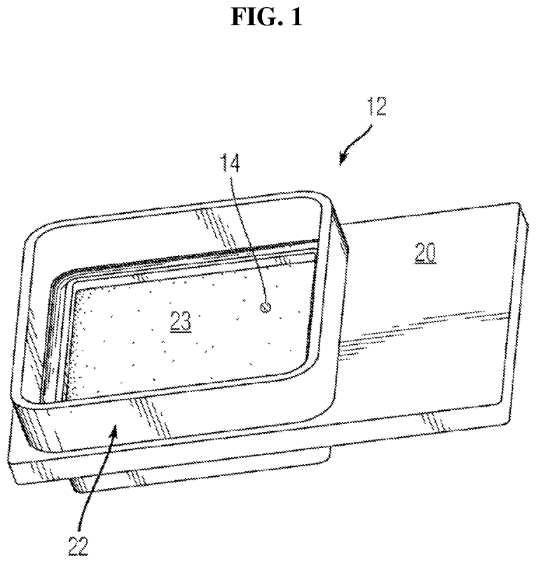

Certain preferred embodiments utilize a carrier, tray, sheet, film, or combinations thereof on which the cationized substrate or base material is deposited. FIG. 1 shows a preferred embodiment of a carrier 12 in the form of a plastic holder with a first planar portion 20 for holding and transporting the carrier 12 and a well 23 containing the base material 14. Well 23 is defined by a raised peripheral wall 22. The carrier or sheet and well may be made of various suitable materials, such as plastic, polyester, amorphous polyester, co-polyester, paper, cotton, wood pulp, wax-coated paper, board, plastic-coated paper, parchment paper, acetate, coated face stock, translucent film, PVC, PET, polypropylene, high density polyethylene, polycarbonate, polyurethane, latex, polystyrene, foam, sponge, rayon, nylon, treated polyester resin film (e.g. mylar), polymer film, acetate or derivatives or combinations thereof, and may further comprise or be composed in whole or in part of synthetic fibers, animal hair, fur, or derivatives or combinations thereof. In preferred embodiments, the carrier, sheet, and/or well are made of a nonporous material such as plastic, polyester, or a polymer film.

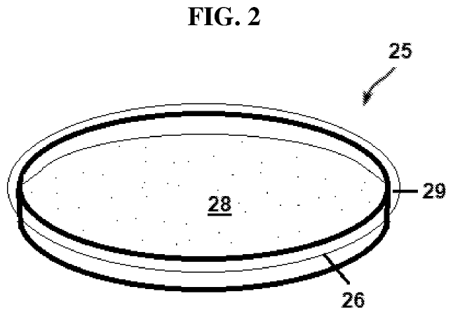

Additional preferred embodiments utilize a substrate or holder for the base material having walls/wells like a cosmetics pan so that dry base material may be pressed into the holder. Wet base material may also be dried into this type of holder if desired. FIG. 2 shows a preferred embodiment of a carrier 25 having walls 26 for holding base material 28. Carrier 25 may be in the form of a metal (usually tin or aluminum) eyeshadow pan with the base material deposited inside but could be made of any suitable material. In the embodiment shown in FIG. 2, a protective film 29 could also optionally be included, to keep the base material 28 dry or protected. In certain preferred embodiments, protective film 29 can be made of a material having UV blocking or sun protection capabilities in order to decrease color fading. In place of, or in addition to, protective film 29, carrier 25 could be accompanied by an envelope or sleeve (not pictured) that carrier 25 is placed in when not in use in order to keep the base material dry or protected. The envelope or sleeve may have UV blocking or sun protection capabilities or may be opaque.

In additional preferred embodiments, the cationized base material may be turned into a slurry or other suitable form and printed or deposited on the sheet or carrier through any suitable means, such as letterpress, offset, gravure, flexography, screenprinting, airbrushing, spray painting, laser printing, drop on demand, continuous inkjet or the like. The slurry may be formed by wetting the base material with a compatible evaporating/volatile solvent that is compatible with the chemistry of the contents of the base materials. This solvent may be used as the dilutant for the cationic ingredient as well if it is compatible. Examples of the solvents may be a volatile liquid such as water, volatile silicone or evaporating solvents such as ethyl alcohol or isopropyl alcohol.

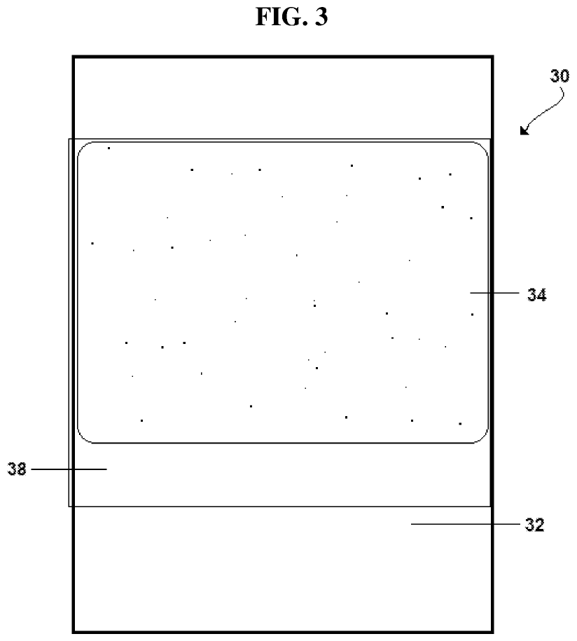

FIG. 3 shows an example of a preferred embodiment of a substrate sheet 30 including a support sheet 32 on which a layer of base material 34 has been deposited. The resulting layer of base material may be about 0.001 mm to about 1 mm in thickness with a generally flat surface, if deposited on a sheet. The generally flat surface enhances the absorption and evenness of the ink and allows the base material to dye evenly without mixing. Loose material may ball up or flocculate when ink is dropped on it, which may lead to uneven and non-uniform coloring. Loose powder is not preferred, but pressed powder may be suitable. A slurry of base material will typically form a flat surface upon drying that is suitable for the present formulations. The printed sheet should be thin enough to allow placement into a printer paper tray or otherwise under the nozzle of a printer, as well as to facilitate ease of storage and transport. In preferred embodiments, the dried base material deposited on the substrate sheet 30 is about 0.10 to about 0.25 g and may have a surface area of about 2.5 inches by 2.5 inches. In certain embodiments, the printed sheet having the base material deposited thereon further comprises a protective film or covering on top of the base material to prevent contamination and disintegration of the base material. A protective film 38 as shown in FIG. 3 could be used to cover the base material. The protective film 38 is removed prior to printing and may be re-affixed to the printed cosmetic layer on the sheet after printing in order to save or store the printed cosmetic for later use. In additional preferred embodiments, a perimeter of reusable/resealable adhesive (not shown in FIG. 3) may be printed on the protected film around the base material. The adhesive can be pressure sensitive, anaerobic, self-crosslinking, U.V. curable, heat curable, or the adhesive material may be dried by evaporation. Coupling of the protective film to the base sheet may be accomplished either with or without the addition of an adhesive as detailed above. Other methods without an adhesive such as hermetic sealing with heat or fusion or sonic sealing, magnets, hook and loop fasteners, or the like may also be used. Protective film 38 can be made of a material having UV blocking or sun protection capabilities in order to decrease color fading. In place of, or in addition to, protective film 38, substrate sheet 30 could be accompanied by an envelope or sleeve (not pictured) that substrate sheet 30 is placed in when not in use in order to keep the base material dry or protected. Any protective covering, such as a protective film, sheet, sleeve, or envelope, can be used. The protective covering may have UV blocking or sun protection capabilities or may be opaque.

The ability of the base material to stay on the sheet without cracking or crumbling is also important. In preferred embodiments, ingredients such as, but not limited to, titanium dioxide, magnesium stearate, zinc stearate, clays, glycerin, silicones, emollients, and other cosmetic additives that make the base material more pliable and that enhance the base material's ability to stick to or stay on the substrate sheet or carrier may be added to the base material formulation or applied to the substrate prior to application of the base material. Turning a dry base powder into a wet slurry and drying it on the carrier is an important part of the process that also helps the base material stick on the carrier.

In preferred embodiments, the dye material can be printed in any combination or pattern, including multiple different shades on a single sheet to allow for sampling, as well as the printing of images or patterns that can be applied directly to the skin. The improved binding between the dye and the base material improves colorfastness on the printed sheet as well as on the skin and with other applied cosmetics.

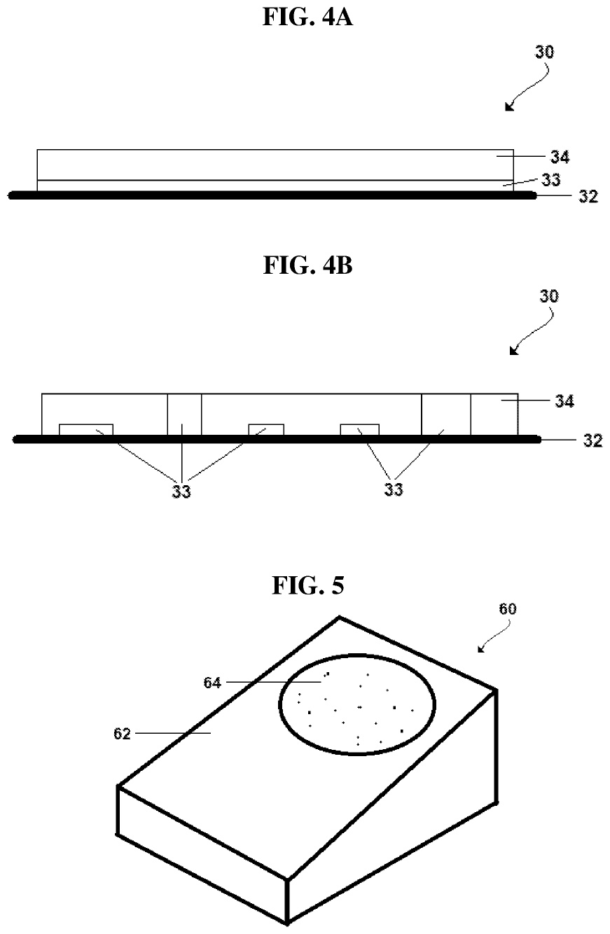

In additional preferred embodiments, a layer of hydrophobic material is first applied to the sheet or carrier and the cationized base material is then deposited in a layer on top of the hydrophobic material. FIG. 4A shows an example of an embodiment of substrate sheet 30 including a support sheet 32 on which a continuous layer of hydrophobic material 33 has been deposited, with a layer of base material 34 deposited on top of or above the hydrophobic material 33. FIG. 4B shows an alternate example of a substrate sheet 30 including a support sheet 32 on which non-continuous portions of hydrophobic material 33 have been deposited or sprinkled onto the support sheet 32, with the base material 34 deposited in a similar fashion on top of the hydrophobic material 33. FIG. 4B shows that the hydrophobic material 33 may extend to the top surface, surrounded by base material 34. The hydrophobic material 33 and base material 34 can be deposited in any patterns. In these embodiments, the hydrophobic material serves as a barrier or guard to retain the dye within the base material. When the colored substrate is about to be removed from the sheet and applied, the user would dip their brush or finger into the colored area and end up mixing the top layer that has the deposited color dye with the bottom hydrophobic material and thereby enhance the waterproofness and colorfastness of the finished product. Additionally, hydrophobic material 33 and base material 34 can be of the same ingredients/substance.

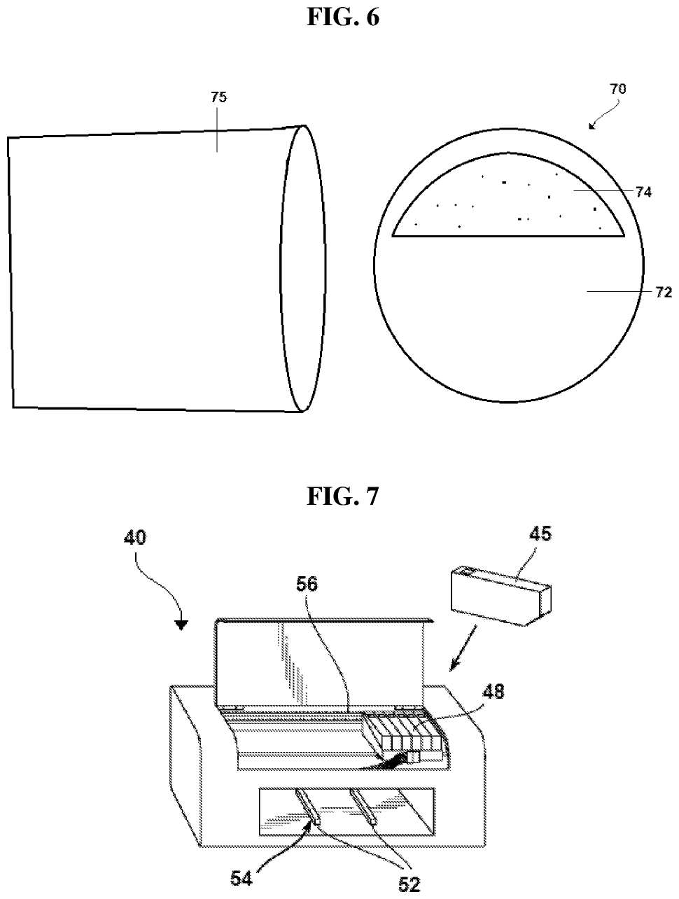

FIG. 5 shows an additional preferred embodiment of a substrate 60 including a support 62 made out of a sponge or foam wedge on which a layer of base material 64 has been deposited. FIG. 6 shows an additional preferred embodiment of a substrate 70 including a support 72 which may be a round cotton pad or wipe on which a layer of base material 74 has been deposited. These figures illustrate how the base material can be deposited in varying positions, amounts, and shapes on a support made of any suitable material. FIG. 6 also shows an envelope or sleeve 75 which can hold substrate 70 when not in use in order to protect base material 74 from drying or UV exposure. Any suitable envelope or sleeve in any suitable shape or material to fit the size and shape of the substrate can be used. The envelope or sleeve can be designed to have an appropriate closure, such as by folding or adhesive.

The carrier or substrate used for liquid, cream, and other non-solid/loose base formulations can preferably be a container with walls or a well such as those shown in FIG. 1 or FIG. 2. If it were on a sheet the base materials might run off. In some embodiments, the container could have an airtight removable film or a lid such as protective film 29 shown in FIG. 2 that could be repositioned and resealed. Some base materials cure by air exposure so it is important to prevent premature curing and to be able to adjust the amount of exposure when applying the cosmetic. If the viscosity of the cream is more on the solid side then depositing on a carrier sheet such as that shown in FIG. 3 is also possible. A ball bearing or the like could be included to assist with mixing. In addition to the resealable film, the carrier could also have an attached applicator or spout. The user could open the resealable film for printing then reseal the film after printing is complete. Then the user could manually evacuate (squeeze, twist-up, or the like) the finished cosmetics composition out of the applicator/spout.

Additional preferred embodiments include a case for storing the substrate or base material. The substrate may be contained in a carrier, which is removably secured to an interior of the case. The carrier may be secured magnetically, with a sliding groove or slot or slit, with a clip, with a wedge, or through any other suitable means to provide a removable connection.

In certain additional preferred embodiments, the cosmetic formulation described herein is used in conjunction with a device for producing a customized cosmetic composition as described in U.S. Pat. No. 9,498,974, incorporated herein by reference. As shown in FIG. 7, this device may be in the form of a printer 40 that is modified to receive and process cosmetic components. The printer 40 may include at least one printer cartridge 45 that may be attached to a printer carriage 48 that moves along a rail 56. The printer 40 may also have an opening 54 for receiving a substrate, such as the substrate sheet or carrier shown in FIG. 1 or 2 including the deposited base material described herein, and allow positioning of the substrate in relation to the printer carriage 48. In this embodiment, guides 52 are included to assist in positioning of the substrate. At least one printer cartridge 45 is provided that contains a dye material as described herein. The printer cartridge 45 is operatively coupled to the printer, such as by means of a print head (not shown), such that the dye material can be applied to the substrate through the print head. The resulting cosmetic composition includes the dye material as it is applied to the base material and is a transferable material that can be removed from the substrate and applied to a part of a human body. The printer 40 may be connected to or otherwise controlled by a computer or any suitable mobile device. As described in U.S. Pat. No. 9,498,974, the computer or mobile device can be programmed with software to control the printer. The software will control the dispensing of the dye material in order to produce the correct selected color at the correct location on the substrate and in the correct amount, based on the type of base material, its total amount or thickness, and its surface area.

In preferred embodiments relating to customized cosmetic formulations, any suitable printer can be used so long as the printer deposits the dye materials described herein that are suitable for use in cosmetic products. In one embodiment, the printer is an inkjet printer which functions as a conventional inkjet printer in that it operates by propelling variably sized droplets of liquid ink/pigment (coloring agent). Ink jet printing, which includes so-called “continuous ink jet” and “drop on demand” technologies, can be used. In these preferred embodiments, the dye material may be contained in one or more cartridges such as those shown in FIG. 7. These cartridges operate in a similar fashion to traditional printer cartridges and contain inks that are suitable for cosmetic applications. For example, the cartridges can contain the following inks: cyan (C), black (K), magenta (M), and yellow (Y). Each cartridge holds only one color or alternatively, one cartridge can contain more than one color with each separated from the other. Some print technology uses more than four cartridges and can include other color cartridges such as, light magenta and light cyan. Depending on whether the printer is of a fixed or disposable head design, the cartridge may contain the print head and nozzle(s) for discharging the ink or there may be a fixed print head in close proximity to the cartridge for receiving and discharging the contents (ink) of the cartridge.

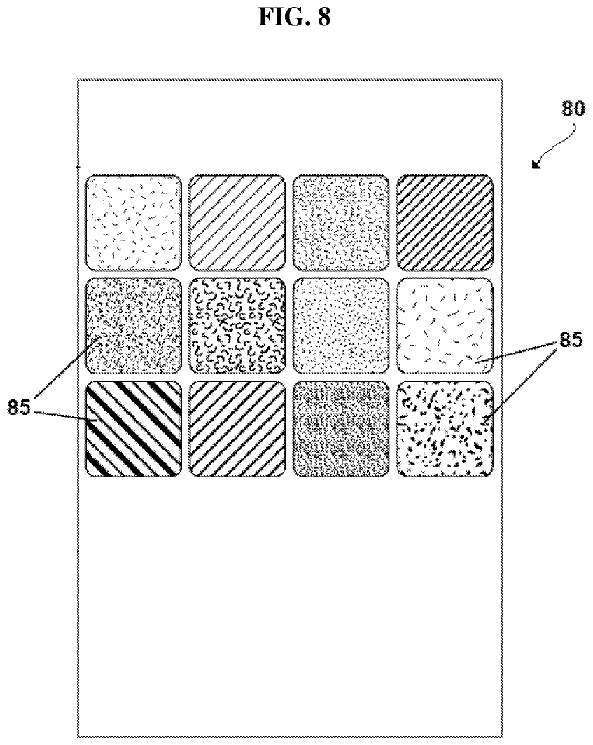

In additional preferred embodiments relating to customized cosmetic formulations, a printer device could be used to print an image using the dye materials onto a non-absorbent sheet that does not contain any substrate or base material. The printed image is maintained in a protected or covered state to allow the ink to stay wet. Then the user could manually stamp or otherwise press the printed image onto a substrate or base material, such that the ink would transfer to the base material in the pattern of the printed image. FIG. 8 shows an embodiment of a transfer sheet 80 on which various dye material portions 85 have been printed, with dye material portions 85 representing a variety of different shades of color. In use, this transfer sheet 80 could be utilized in conjunction with the substrate sheet 30 shown in FIG. 3. The user could remove protective film 38 from substrate sheet 30 and press the transfer sheet 80 against substrate sheet 30, such that dye material portions 85 contact base material 34 and are transferred to the base material to produce dyed base material for use as cosmetics. In certain embodiments, protective film 38 could then be applied to the substrate sheet 30 to protect the dyed cosmetic material.

Preferred embodiments described herein include a method for preparing a customized cosmetic, comprising using a printer to apply ink in at least one selected color and a selected pattern or shape onto an undyed substrate material, where the undyed substrate material is prepared by mixing ingredients comprising mica, polyquaternium-6, water, and a preservative into a slurry, spraying the slurry onto a carrier, and drying the slurry to produce the undyed substrate material. The ingredients in the undyed substrate material may further comprise triethoxysilylpropyl acetyl hydroxyprolinate. The preservative is preferably a combination of phenethyl alcohol, caprylhydroxamic acid, and glycerin. In preferred embodiments, the ingredients mixed to prepare the undyed substrate material comprise about 70% to about 90% by weight, preferably about 80% by weight, of a combination of mica and triethoxysilylpropyl acetyl hydroxyprolinate, where the combination includes mica in about 98-99% by weight and triethoxysilylpropyl acetyl hydroxyprolinate in about 1-2% by weight. The mixture made to prepare the undyed substrate material preferably further contains about 10% to about 30% by weight, preferably about 20% by weight, of a solution which includes water, about 1% to about 3% polyquaternium-6 by weight, preferably about 2% polyquaternium-6 by weight, and about 1% of a preservative. The water can be deionized water, distilled water or regular tap water. The preservative is preferably made up of about 42-60% phenethyl alcohol, by weight, about 12-18% caprylhydroxamic acid, by weight, and about 28-40% glycerin, by weight. After mixing, the slurry will contain about 0.05% to about 0.90% polyquaternium-6 by weight and about 0.10% to about 0.30% preservative by weight, with the remainder being the water mixed with the combination of mica and triethoxysilylpropyl acetyl hydroxyprolinate. After drying, the substrate material will contain about 0.06% to about 1.26% polyquaternium-6, by weight, and about 0.11% to about 0.42% of a preservative, by weight, with the rest being the combination of mica and triethoxysilylpropyl acetyl hydroxyprolinate. The undyed substrate material is preferably between about 0.001 mm and 1 mm in thickness on the carrier. The carrier is preferably any suitable nonporous material and can be plastic, polyester, or a polymer film. The ink is any suitable dye formulation that is approved for use in cosmetics, including those in Table 2 below. The printer is preferably controlled by an application or software that allows a user to select images, patterns, and/or colors for printing onto the undyed substrate material. In preferred embodiments, the method for preparing a customized cosmetic further comprises the step of covering the customized cosmetic with a protective covering after the ink is applied. The protective covering, which may be a film, sheet, envelope, or sleeve, is preferably made of a material that blocks UV radiation, has sun protection capabilities, or is opaque.

Additional preferred embodiments described herein include a system for preparing a customized cosmetic comprising ink, where the ink is any suitable dye formulation that is approved for use in cosmetics, including those in Table 2 below, a printer, an application or software for controlling the printer, and one or more carriers on which modified undyed substrate material has been deposited, where the printer applies the ink to the modified undyed substrate material, and wherein the application or software directs the printer to use the ink to apply a selected image, pattern, or color to the modified undyed substrate material, to produce a customized cosmetic.

### Example 1. Exemplary Formulations and Performance

In preferred embodiments described herein, and in the examples described below, the following exemplary dye formulations were used. These were SensiJet® FSE (Sensient Imaging Technologies, Switzerland) edible inks.

TABLE 2

| US | EU | | | | |
|---|---|---|---|---|---|
| MAGENTA | MAGENTA | CYAN | YELLOW | BLACK | CLEANER |
| Water | Water | Water | Water | Water | Water |
| Propylene | Propylene | Propylene | Propylene | Propylene | Propylene |
| Glycol | Glycol | Glycol | Glycol | Glycol | Glycol |
| Glycerin | Carmoisine | Glycerin | Glycerin | FD&C Red No. | Glycerin |
| | | | | 40 | |
| FD&C Red No. | Glycerin | FD&C Blue | FD&C Yellow | Glycerin | Polysorbate 80 |
| 3 | | No. 1 | No. 5 | | |
| Polysorbate 80 | Polysorbate 80 | Polysorbate 80 | Polysorbate 80 | FD&C Blue | Methyl Paraben |
| | | | | No. 1 | |
| FD&C Red No. | Mono and Di- | Mono and Di- | Mono and Di- | Polysorbate 80 | Mono and Di- |
| 40 | Glycerides | Glycerides | Glycerides | | Glycerides |
| Sodium | Potassium | Sodium | FD&C Yellow | FD&C Yellow | Sodium |
| Hydroxide | Citrate | Hydroxide | No. 6 | No. 6 | Hydroxide |
| Mono and Di- | Sodium | | Sodium | Mono and Di- | |
| Glycerides | Hydroxide | | Hydroxide | Glycerides | |
| FD&C Blue | | | | Sodium | |
| No. 1 | | | | Hydroxide | |

In these examples, the dye formulations were not modified to include a cationic or anionic component. These dyes already possess a general anionic (negative) charge due to their formulations.

Several base formulations or undyed cosmetic materials were also tested for dye retention with and without a cationic ammonium treatment of polyquaternium-6 (“P6”) and/or a modified cationized clay compound. Exemplary tested formulations are noted below in Table 3. Base formulations noted below in Table 3, to the extent that more than one ingredient was included, were mixed together manually until the ingredients were uniformly combined. If P6 was added it was added in a solution of distilled water. For each tested formulation, approximately 0.2 g-2 g of undyed base formulation was portioned onto non-absorbent materials such as freezer paper, parchment paper, or polyester. Each base formulation was tested with solutions containing different concentrations of P6 (none, 0.75%, 1%, 2%). High percentages such as 20% P6 were eliminated early on since they make the base materials unusable (gummy). If the base material was treated with P6, a MLA Pipette DIGITAL (VistaLab Technologies Brewster, N.Y.), was used to deposit approximately 0.2-1 ml of solution onto the base material. Usually enough solution was added so that the mixture turned into a liquid/paint-like consistency. The solution and base material were then mixed thoroughly until the mixture was homogenous and it was left to dry overnight.

The base formulations from Table 3 below were mixed with distilled water to form a slurry and were deposited on a sheet of polyester or plastic-coated paper and allowed to dry. The sheets specifically included plastic coated freezer paper (with a polyethylene coating) (“Freezer Paper”) and a light/optical diffuser film (“Polyester”). The deposited base formulation formed a layer of less than 1 mm. A combination of the cyan and yellow dyes from Table 2 above was then applied to the base material. The dye was deposited with a MLA Pipette DIGITAL (VistaLab Technologies Brewster, N.Y.). 2-20 μL of ink was deposited on the base material and smeared/stamped with a small plastic stick or felt to mimic the depositing behavior of a inkjet printhead. The ink was allowed to dry for at least 1-5 minutes before application to prepared skin for testing.

The prepared cosmetic sample, made up of the dyed base material, was then applied to a portion of skin pre-treated with a face foundation cosmetic. Two portions of 200 μL of water were then poured over the applied cosmetic sample and a visual evaluation of the cosmetic samples was made. Color bleeding, color staining, color change, and edging were visually evaluated. Edging occurs when lines/edges are created in the formulation where the water edge traversed. The cosmetic samples were graded on a scale with regard to their performance as (1) Excellent, (2) Best, (3) Good, and (4) Poor. Excellent samples showed no bleeding, staining, color change, or edging. Best samples showed either no or few instances of light bleeding, staining, color change, and/or edging. Good samples showed more instances of light bleeding, staining, color change, and/or edging. Samples that showed consistent light or heavier bleeding, staining, color change, and/or edging were considered to be Poor. The results of the evaluation for the base material making up each cosmetic sample is shown in the rightmost column of Table 3 below.

TABLE 3

| | Base Formulation | Base Formulation | | |
|---|---|---|---|---|
| No. | Trade Name (if any) | Components | Cationic Treatment | Performance |
| 1A | KOBO | Rutile titanium dioxide | None | Poor |
| 1B | RBTD/MM3 (Kobo | coated with magnesium | P6, 2% | Good |
| | Products, Inc, South | myristate at about 2.5-3.5 | | |
| | Plainfield, NJ) | weight % | | |
| 2A | TKB Magnesium | Mica (94.5% by weight), | None | Poor |
| 2B | Myristate Mica | magnesium myristate | P6, 1% | Good |
| 2C | (TKB Trading, | (5.5% by weight), after | P6, 2% | Best |
| 2D | Oakland, CA) | reaction of myristic acid | P6, 1%, and | Excellent |
| | | and magnesium | disteardimonium | |
| | | hydroxide | hectorite, | |
| | | | cyclopentasiloxane, | |
| | | | and specially | |
| | | | denatured (SD) | |
| | | | alcohol¹, about 1:1 | |
| | | | with base formulation | |
| | | | by weight | |
| 3A | Signature Mineral | Mica (20-50%), titanium | None | Poor |
| 3B | Base MS | dioxide (5-30%), zinc | P6, 1% | Best |
| 3C | (MakingCo smetics, | oxide (10-40%), | P6, 2% | Best |
| 3D | Redmond, WA) | magnesium stearate (5- | P6, 1%, and | Excellent |
| | | 30%), silica (5-30%), | Quaternium-18 | |
| | | amounts vary per batch | Hectorite, about 1:1 | |
| | | | with base formulation | |
| | | | by weight | |
| 3E | | | Disteardimonium | Excellent |
| | | | hectorite, | |
| | | | cyclopentasiloxane, | |
| | | | and specially | |
| | | | denatured (SD) | |
| | | | alcohol¹, about 1:1 | |
| | | | with base formulation | |
| | | | by weight | |
| 3F | | | P6, 1%, and | Excellent |
| | | | disteardimonium | |
| | | | hectorite, | |
| | | | cyclopentasiloxane, | |
| | | | and specially | |
| | | | denatured (SD) | |
| | | | alcohol¹, about 1:1 | |
| | | | with base formulation | |
| | | | by weight | |
| 4A | TKB Extender | Mica (54-59%), Titanium | None | Poor |
| 4B | White (TKB | dioxide (41-46%), | P6, 0.75% | Good |
| 4C | Trading) | amounts vary per batch | P6, 2% | Best |
| 5A | Signature Mineral | Mica (20-50%), titanium | None | Poor |
| 5B | Base (Making | dioxide (5-30%), zinc | P6, 1% | Good |
| | Cosmetics) | oxide (10-40%), silica (5- | | |
| | | 30%), amounts vary per | | |
| | | batch | | |
| 6A | TKB Matte Texture | Titanium dioxide (10- | None | Poor |
| 6B | Base (TKB Trading) | 12%), mica (74-76%), | P6, 1% | Good |
| | | magnesium stearate (10- | | |
| | | 12%), amounts vary per | | |
| | | batch | | |
| 7A | Soft-Tex Titanium | Anatase, oil soluble | P6, 1% | Poor |
| 7B | Dioxide (Sun | titanium dioxide | P6, 2% | Best |
| | Chemical, New | | | |
| | Jersey) | | | |
| 8A | KOBO BTD/MM3 | Titanium dioxide coated | None | Poor |
| 8B | (Kobo Products, | with magnesium | P6, 1% | Good |
| | Inc.) | | | |
| | | myristate at about 2.5-3.5 | | |
| | | weight % | | |
| 9A | Boron Nitride (TKB | Boron nitride | None | Poor |
| 9B | Trading) | | P6, 0.75% | Poor |
| 9C | | | P6, 1% | Good |
| 10A | Bentonite (Making | Bentonite | None | Poor |
| 1OB | Cosmetics) | | P6, 1% | Good |
| 11A | NobleThix R PC | Trihydroxystearin | None | Poor |
| 11B | (Noble Roots, | | P6, 1% | Good |
| | Lawrenceville, GA) | | | |
| 12A | Bentone Gel PIO V | Hydrogenated | None | Best |
| | (Elementis | polyisobutene, | | |
| | Specialties, London, | disteardimonium | | |
| | UK) | hectorite, propylene | | |
| | | carbonate | | |
| 13A | Bentone Gel VS-5 V | Disteardimonium | None | Best |
| | HV (Elementis | hectorite, | | |
| | Specialties) | cyclopentasiloxane, and | | |
| | | specially denatured (SD) | | |
| | | alcohol | | |
| 14A | Bentone Gel 38 V | Disteardimonium | None | Best |
| | CG (Elementis | hectorite | | |
| | Specialties) | | | |
| 15A | Sumecton San-P | Quaternium-18 | None | Best |
| | (Kobo Products, | | | |
| | Inc.) | | | |
| 16A | TIXOGEL MP 250 | Stearalkonium bentonite | None | Best |
| 16B | (Eckart America, | | P6, 1% | Best |
| | Louisville, KY) | | | |
| 17A | | Quaternium-90 bentonite | None | Best |
| 17B | TIXOGEL VP V | | P6, 1% | Best |
| | (Eckart America) | | | |
| 18A | TIXOGEL VZ V | Stearalkonium bentonite | None | Best |
| 18B | (Eckart America) | | P6, 1% | Best |
| 19A | TIXOGEL LG M | Stearalkonium bentonite | None | Best |
| 19B | (Eckart America) | | P6, 1% | Best |

¹Bentone Gel VS-5 V HV (Ethyl Alchohol 2.5-10%, Octa methylcyclotetrasiloxane <1%), Elementis Specialties, London, UK

It can be seen from the results above that the Excellent performing base materials included either a cationic ammonium ingredient, namely polyquaternium-6, at about 1% by weight, in combination with a modified cationized clay compound (e.g. 3E, 3F, and 2D), or a modified cationized clay compound without a cationic ammonium ingredient (e.g., 2D). Several of the Best performing base materials included a modified cationized clay compound without a cationic ammonium ingredient (e.g. 12A, 13A, 14A, and 15A). Additional Best performing materials included a modified cationized clay compound and had similar performance with (e.g., 16B, 17B, 18B, and 19B) or without (e.g., 16A, 17A, 18A, and 19A) a cationic ammonium ingredient (P6) added as well. Of the base formulations that showed Best performance that did not include a modified cationized clay material (e.g., 7A, 3C, 3B, 4C, and 2C), these formulations included titanium dioxide or a magnesium-containing component or mica, or combinations thereof.

While not reflected in Table 3 above, certain powdered starches were also tested. Although powdered starches such as arrowroot, corn, tapioca, rice, and quinoa showed poor performance overall, the best performing base material in this category was quinoa which contained much higher levels of magnesium, iron and protein than the other starches.

Notably, all of the base formulations prepared and tested in Table 2 above that did not include either a cationic ammonium ingredient (P6) or a modified cationized clay compound showed Poor performance.

### Example 2. Performance Variables

Additional evaluations were performed relating to the cosmetic formulations.

Material Used as Sheet for Depositing Base Formulations

In conjunction with the evaluation of the base formulations in Table 3 above, certain base formulations were formed into slurries and deposited on either Polyester sheets or Freezer Paper sheets. Of the Excellent performing base formulations, 3D performed equally well on polyester and freezer paper. 3E, 3F, and 2D were all tested on polyester sheets and showed Excellent performance. For 7B (titanium dioxide) and 2C (magnesium myristate mica), the Best performance occurred on polyester, and these samples showed reduced performance on freezer paper. Samples 3C, 3B, and 4C all showed Best performance on freezer paper, with reduced performance on polyester.

This data does not indicate a particular trend and would not impact a base formulation that is not made into a slurry and deposited on a sheet.

Non-Cationized Clays

Similar performance tests were carried out using non-cationized clay materials, including bentonite, hectorite, and hectorite with hydroxyethylcellulose. These clay materials were not modified with any cationic ingredients. All of the non-cationized clays performed poorly and demonstrated undesirable results including clumping, color change, and swelling.

Titanium Dioxide Performance

Titanium dioxide is available as water dispersible and oil dispersible. Water dispersible titanium dioxide blends easily into water and oil dispersible titanium dioxide blends easily into oil. The difference lies in the amount of salts and minerals in the product. More salt makes the titanium dioxide more dispersible in water and less salt makes it more dispersible in oil. Titanium dioxide is also available as anatase and rutile. Anatase is less hard (5.5-6 vs. 6-6.5 Mohs) and dense (specific gravity about 3.9 vs. 4.2). Also, anatase is optically negative whereas rutile is positive, and its luster is even more strongly adamantine or metallic-adamantine than that of rutile. Both the particle size distribution and surface charge associated with the pigments can be controlled, eliminating variability and instability. The pigments are smaller in size with a more uniform particle size distribution. The anatase (oil soluble) titanium dioxide samples of 7A and 7B in Table 2 above were tested on both polyester and freezer paper and showed better performance on polyester. The 2% concentration of P6 also performed better on both materials. Anatase oil soluble titanium dioxide (TKB) was also tested with 1% and 2% P6 by weight on both polyester and freezer paper. Performance was better on polyester and was better at 1% rather than 2% P6. Rutile titanium dioxide was also tested with 1% and 2% P6 by weight on both polyester and freezer paper. I's performance was comparable to TKB titanium dioxide anatase oil soluble, and showed better performance at 1% on Freezer Paper, however it was the least effective of the titanium dioxides.

Emulsifiers

The base formulations 4A and 5A from Table 3 above, which showed Poor performance in the absence of P6, were modified with 1% of an emulsifier—either Polysorbate 20 or Polysorbate 80. The same performance tests were carried out as described above. The formulations all showed Poor performance with confirmed color bleeding, stain, change, and edging. Accordingly, using emulsifiers without a cationic treatment does not improve performance.

Cationic Ammonium Ingredients

Other cationic ammonium ingredients besides polyquaternium-6 were tested. Base formulation 4A from Table 3 was modified alternately with 1% of the cationic ingredients quaternium-31 (dicetyldimonium chloride, isopropyl alcohol) or cetrimonium chloride (with water). Performance was considered Poor. Base formulations 4A and 5A were also modified individually with polyquaternium-51, quaternized honey SA, quaternized honey PF, or Poly Suga Quat L-1010P (polyquaternium-78), in each instance on an “as needed” basis. Performance was still considered Poor.

Anionic, Amphoteric, and Non-Ionic Ingredients

Base formulation 4A from Table 3 was modified alternately with 5% of the emulsifier cocamidopropyl betaine (coco betaine, a mild amphoteric surfactant), sodium lauryl sulfate (an anionic surfactant), or a blend of decyl glucoside and sodium lauroyl lactylate (a non-ionic mild surfactant blend). In each case, performance was considered Poor.

5% P6 Solutions

The base formulations shown in Table 3 as 3A, 4A, and 7A were tested further to compare the effects of treatment with 1%, 2%, 3%, and 5% P6 solutions. 0.20 g of base material was treated with 1.0 g-2.0 g of the designated P6 solution. The same performance tests were carried out. For 3A, the best performance was at 1% and 2%, with the worst performance at 5%. For 4A, the best performance was at 2% and the worst performance was at 5%. For 7A, the best performance was at 3% and the worst performance was at 5%. This shows that the use of 5% P6 solutions to treat the base formations does not produce effective results.

### Example 3. Color Stability Testing

Different base formulations were prepared and tested for their ability to resist color fading. Each tested formulation included wet ingredients made up (by weight percent) of 96.5% water, 2% P6, 1% glycerine, and 0.5% phenethyl alcohol, with glycerin and caprylhydroxamic acid (preservative). The dry ingredients for each tested formulation are listed below. For each tested formulation, 0.6 g of dry ingredients was mixed with 2.4 g of wet ingredients. The resulting slurry was thinly distributed on a polyester/mylar/acetate film and allowed to dry. An image was then printed on each dried substrate using a printer containing dyes identified above in Table 2. The printed images on the substrates were then exposed to sunlight near a window for three days.

TABLE 4

| No. | Dry Ingredients | Performance |
|---|---|---|
| 1 | 20-30% titanium dioxide | Poor |
| | 70-80% Mica | |
| 2 | 100% Mica | Poor |
| 3 | 99% Mica | Best |
| | 1% sodium gluconate | |
| 4 | 89% Mica | Good |
| | 10% HDI/trimethylol hexyllactone | |
| | crosspolymer with methyl methacrylate | |
| | crosspolymer | |
| | 1% sodium gluconate | |

### Example 3. Conductivity/Resistivity

Different base formulations were prepared and tested for their conductivity and resistivity. Resistance (S2) was measured with a Sperry DM-4400A 3½ Digit Handheld Digital Multimeter. Conductivity (0/cm) was measured with OHAUS ST-20M-B pH, Conductivity, Temperature, TDS Water Analysis Pen Meter (Conductivity Range: 0-1999 μS/cm). Each tested formulation included a base material formulation mixed with a solution containing a selected concentration of P6 (0%, 1%, 2%, 3%, 4%, 5%). All solutions were 25 g in weight, including the P6 and deionized water (type II, final filtered at 0.2 μS/cm conductivity). The resistance measurements for each solution of P6 are shown below in Table 5.

TABLE 5

| P6 % | KΩ |
|---|---|
| 0 | 835 |
| 1 | 330 |
| 2 | 120 |
| 3 | 133 |
| 4 | 200 |
| 5 | 295 |

Two different base materials were used. Base A was the base formulation labeled as 5A in Table 3 above. It included Mica (20-50%), titanium dioxide (5-30%), zinc oxide (10-40%), silica (5-30%). Base B included Mica (98-99%) and Triethoxysilylpropyl Acetyl Hydroxyprolinate (1-2%). For each tested base, 1.5 grams of dry base formulation was added to 6 grams of each P6 solution (0%, 1%, 2%, 3%, 4%, 5%) and the materials were mixed together manually until the ingredients were uniformly combined and homogenous. Conductivity/Resistance measurements were taken while the mixtures were still in wet, slurry form. The results are shown in Tables 6 and 7 below.

TABLE 6
Solution + Base A (Wet)

| P6 % | kΩ | μS/cm |
|---|---|---|
| 0 | 2.3 MΩ | 126 |
| 1 | 1080 | 540 |
| 2 | 740 | 1365 |
| 3 | 927 | 1140 |
| 4 | 713 | OOO |
| 5 | 663 | OOO |

TABLE 7
Solution + Base B (Wet)

| P6 % | μS/cm |
|---|---|
| 0 | 129 |
| 1 | 629 |
| 2 | 1505 |
| 3 | 1400 |
| 4 | 1700 |
| 5 | OOO |

The slurries were then deposited in amounts of about 0.2-2 g on sheets of coated, non-absorbent paper and allowed to dry overnight. The dry deposited base material formed a layer of less than 1 mm. Conductivity/Resistance measurements were taken. Then, a combination of the cyan and yellow dyes from Table 2 above was applied to the base material. The dye was deposited with a MLA Pipette DIGITAL (VistaLab Technologies Brewster, N.Y.). 2-20 μL of ink (Sensijet) was deposited on the base material and smeared/stamped with a small plastic stick or felt to mimic the depositing behavior of a inkjet printhead. The ink was allowed to dry for at least 1-5 minutes before application to prepared skin for testing. Testing was carried out as described above with regard to the evaluations in Table 3. The resistance measurements for the dyes are shown in Table 8 below. The results of the Conductivity/Resistance measurements and the dye retention testing for the base formulations are shown in Tables 9 and 10 below.

TABLE 8

| Ink | kΩ |
|---|---|
| Yellow | 550-650 |
| Magenta | 650-750 |
| Cyan | 800-900 |

TABLE 9
Base A (Dry)

| | | Ink |
|---|---|---|
| | | Retention |
| P6 %* | kΩ | Result |
| 0 | 0 | Poor |
| 1 | 700-1300 | Best |
| 2 | 600-800 | Best |
| 3 | 300-600 | Excellent |
| 4 | 900-1000 | Poor |
| 5 | 16-19 MΩ | Poor |

*P6% reflects % used in solution, prior to drying.

TABLE 10
Base B (Dry)

| | | Ink |
|---|---|---|
| | | Retention |
| P6 %* | kΩ | Result |
| 0 | 0 | Poor |
| 1 | 0 | Excellent |
| 2 | 9-10 MΩ | Excellent |
| 3 | 6-7 MΩ | Poor |
| 4 | 2-3 MΩ | Poor |
| 5 | 3-4 MΩ | Poor |

*P6% reflects % used in solution, prior to drying.

The results essentially confirm that the amounts of P6 that are most preferred in the solutions mixed with the base formulations are amounts that range from about 1% to about 3%. Base B in particular showed poor performance at 0% P6 but excellent performance when using a 1-2% P6 solution. Base formulations mixed with solutions of 5% P6 did not produce effective results with regard to ink retention. Further, there is no particular correspondence between conductivity and/or resistance and the performance of the base formulation in the retention test.

### REFERENCES

The contents of the following are incorporated herein by reference:
U.S. Pat. No. 9,498,974
WO 2015/186583
WO 2014/135915
U.S. Patent Application Publication No. 2018/0027950

What is claimed:

1. A modified undyed substrate material for use in preparing customized cosmetics, comprising:
one or more polar ingredients, wherein the one or more polar ingredients comprise one or more cationic ammonium ingredients, one or more modified cationized clay compounds, one or more ionized salts comprising a cation having a +2, +3, or +4 charge, or combinations thereof; and
base material comprising mica, wherein the modified undyed substrate material comprises an electrostatic charge.

2. The modified undyed substrate material of claim 1, wherein the one or more polar ingredients is polyquaternium-6.

3. The modified undyed substrate material of claim 2, wherein the modified undyed substrate material comprises about 0.06% to about 1.26% polyquaternium-6 by weight.

4. The modified undyed substrate material of claim 1, wherein the one or more cationic ammonium ingredients comprise polyquaternium-6, polyquaternium-7, polyquaternium-10, quaternium-31, cetrimonium chloride, polyquaternium-51, guar hydroxypropyltrimonium chloride, cocodimonium hydroxypropyl hydrolyzed rice protein or combinations thereof.

5. The modified undyed substrate material of claim 1, wherein the one or more modified cationized clay compounds comprise quaternium-18 hectorite, disteardimonium hectorite, stearalkonium bentonite or combinations thereof.

6. The modified undyed substrate material of claim 1, wherein the one or more ionized salts comprising a cation having a +2, +3, or +4 charge comprise ionized salts comprising a cation of magnesium, barium, or calcium.

7. The modified undyed substrate material of claim 1, wherein the base material further comprises a magnesium-containing component.

8. The modified undyed substrate material of claim 1, wherein the base material further comprises triethoxysilylpropyl acetyl hydroxyprolinate, titanium dioxide, magnesium stearate, zinc stearate, magnesium myristate, magnesium hydroxide, myristic acid, zinc oxide, silica, boron nitride, trihydroxystearin, bentonite, or combinations thereof.

9. The modified undyed substrate material of claim 1, wherein the base material further comprises triethoxysilylpropyl acetyl hydroxyprolinate in an amount of about 1% to about 2% by weight of the base material.

10. The modified undyed substrate material of claim 1, wherein the base material further comprises a preservative, and wherein the preservative comprises phenethyl alcohol, caprylhydroxamic acid, and glycerin.

11. The modified undyed substrate material of claim 1, wherein the electrostatic charge is a positive charge.

12. The modified undyed substrate material of claim 1, further comprising a carrier on which the modified undyed substrate material is deposited.

13. The modified undyed substrate material of claim 12, wherein the modified undyed substrate material is deposited on the carrier in a layer of about 0.001 mm to about 1 mm in thickness.

14. A method for preparing a modified undyed substrate material, comprising:
preparing a base material, wherein the base material comprises mica;
mixing the base material with a solution comprising one or more cationic ammonium ingredients to produce a slurry, wherein the cationic ammonium ingredients are present in the solution at a concentration ranging from about 1% to about 3%;
depositing the slurry on a carrier; and
allowing the slurry to dry to produce a modified undyed substrate material, wherein the modified undyed substrate material comprises a positive electrostatic charge.

15. The method of claim 14, wherein the one or more cationic ammonium ingredients comprise polyquaternium-6.

16. The method of claim 14, wherein the base material further comprises triethoxysilylpropyl acetyl hydroxyprolinate and a preservative.

17. The method of claim 14, wherein the slurry is deposited on the carrier in a layer of about 0.001 mm to about 1 mm in thickness.

18. A kit for preparing a customized cosmetic, comprising:
a modified undyed substrate material deposited on a carrier in a layer of about 0.001 mm to about 1 mm in thickness, wherein the modified undyed substrate material comprises one or more polar ingredients, wherein the one or more polar ingredients comprise one or more cationic ammonium ingredients, one or more modified cationized clay compounds, one or more ionized salts comprising a cation having a +2, +3, or +4 charge, or combinations thereof, and a base material comprising mica, wherein the modified undyed substrate material comprises an electrostatic charge; and
a dye material, wherein the dye material comprises a second electrostatic charge, wherein the first electrostatic charge is opposite to the second electrostatic charge, and wherein the dye material is applied to the modified undyed substrate material to produce a customized cosmetic.

19. The kit of claim 18, wherein the first electrostatic charge is positive and the second electrostatic charge is negative.

20. The kit of claim 18, wherein the one or more polar ingredients is polyquaternium-6.

21. The kit of claim 20, wherein the modified undyed substrate material comprises about 0.06% to about 1.26% polyquaternium-6 by weight.

22. The kit of claim 18, wherein the one or more cationic ammonium ingredients comprise polyquaternium-6, polyquaternium-7, polyquaternium-10, quaternium-31, cetrimonium chloride, polyquaternium-51, guar hydroxypropyltrimonium chloride, cocodimonium hydroxypropyl hydrolyzed rice protein or combinations thereof.

23. The kit of claim 18, wherein the one or more modified cationized clay compounds comprise quaternium-18 hectorite, disteardimonium hectorite, stearalkonium bentonite or combinations thereof.

24. The kit of claim 18, wherein the one or more ionized salts comprising a cation having a +2, +3, or +4 charge comprise ionized salts comprising a cation of magnesium, barium, or calcium.

25. The kit of claim 18, wherein the base material further comprises a magnesium-containing component.

26. The kit of claim 18, wherein the base material further comprises triethoxysilylpropyl acetyl hydroxyprolinate, titanium dioxide, magnesium stearate, zinc stearate, magnesium myristate, magnesium hydroxide, myristic acid, zinc oxide, silica, boron nitride, trihydroxystearin, bentonite, or combinations thereof.

27. The kit of claim 18, wherein the base material further comprises triethoxysilylpropyl acetyl hydroxyprolinate in an amount of about 1% to about 2% by weight of the base material.

28. The kit of claim 18, wherein the base material further comprises a preservative, and wherein the preservative comprises phenethyl alcohol, caprylhydroxamic acid, and glycerin.

29. The kit of claim 18, further comprising a transfer sheet, wherein the dye material is deposited on the transfer sheet.

30. The kit of claim 18, further comprising a printer cartridge, wherein the dye material is contained in the printer cartridge.

31. The kit of claim 30, further comprising a printer device, wherein the printer device is configured to receive the printer cartridge and apply the dye material to the modified undyed substrate material.

32. A substrate for preparing a customized cosmetic, comprising:
a carrier, wherein the carrier comprises plastic, polyester, or a polymer film;
a modified undyed substrate material deposited on the carrier in a layer of about 0.001 mm to about 1 mm in thickness, wherein the modified undyed substrate material comprises polyquaternium-6 in an amount of about 0.06% to about 1.26% by weight, a base material, wherein the base material comprises about 98% to about 99% mica by weight and about 1% to about 2% triethoxysilylpropyl acetyl hydroxyprolinate by weight, and a preservative, wherein the preservative comprises phenethyl alcohol, caprylhydroxamic acid, and glycerin, and wherein the modified undyed substrate material comprises a positive electrostatic charge; and
a protective covering, wherein the protective covering is placed over the modified undyed substrate material, and wherein the protective covering is comprised of materials that block UV radiation.

33. A method for preparing a customized cosmetic, comprising:
selecting images, patterns, or colors for printing onto a substrate to prepare a customized cosmetic;
transmitting instructions to a printer to print selected images, patterns, or colors onto the substrate;
using the printer to apply ink onto the substrate to form the selected images, patterns, or colors and produce the customized cosmetic, wherein the substrate comprises a carrier and a modified undyed substrate material deposited on the carrier in a layer of about 0.001 mm to about 1 mm in thickness, wherein the modified undyed substrate material comprises one or more polar ingredients, wherein the one or more polar ingredients comprise cationic ammonium ingredients, one or more modified cationized clay compounds, one or more ionized salts comprising a cation having a +2, +3, or +4 charge, or combinations thereof, and a base material comprising mica, wherein the modified undyed substrate material and the ink comprise opposite electrostatic charges.

34. The method of claim 33, wherein the modified undyed substrate material comprises polyquaternium-6 in an amount of about 0.06% to about 1.26% by weight, a base material, wherein the base material comprises about 98% to about 99% mica by weight and about 1% to about 2% triethoxysilylpropyl acetyl hydroxyprolinate by weight, and a preservative, wherein the preservative comprises phenethyl alcohol, caprylhydroxamic acid, and glycerin.
